(19) United States
(12) Patent Application Publication
(10) Pub. No.: US 2026/0367073 A1
(43) Pub. Date: Nov. 19, 2026

(54) DNA METHYLATION DETECTION METHOD USING ENZYME-BASED CHROMOSOME PAINTING

(71) Applicant: SEOUL NATIONAL UNIVERSITY R&DB FOUNDATION, Seoul (KR)

(72) Inventors: Jin Hoe HUH, Seoul (KR); Woo Lee CHOI, Seoul (KR); Min Jeong KIM, Seoul (KR)

(21) Appl. No.: 19/138,324

(22) PCT Filed: Dec. 13, 2023
(86) PCT No.: PCT/KR2023/020513

§ 371 Date: Jun. 12, 2025

(30) Foreign Application Priority Data
Dec. 13, 2022 (KR) 10-2022-0174136
Jun. 14, 2023 (KR) 10-2023-0076316

(51) Int. Cl. G01N 33/53; C12Q 1/26; C12Q 1/6806; G01N 1/30

(52) U.S. Cl. CPC G01N 33/5308; C12Q 1/26; C12Q 1/6806; G01N 1/30; G01N 2333/90245

(57) ABSTRACT

One aspect relates to a method of detecting DNA methylation sites within a chromosome by replacing DNA methylation sites of the chromosome with fluorescently labeled nucleotides, wherein the method, by targeting fixed chromatin, not only enables identification of the location and density of methylated DNA in the chromosome, but also facilitates chromatin region identification and epigenetic analyses through compatibility with various conventional immunofluorescence labeling techniques.

### TECHNICAL FIELD

The present disclosure relates to a method of detecting DNA methylation sites using enzymes.

### BACKGROUND

DNA methylation is a reaction in which a methyl group is transferred to the 5-carbon position of cytosine in DNA by a DNA methyltransferase, converting it to 5-methylcytosine (5mC). DNA methylation primarily induces the formation of heterochromatin, which is accompanied by chromatin condensation, and results in the suppression of genes. DNA methylation plays an important role in the development, differentiation, and growth of eukaryotic organisms, and abnormal DNA methylation may contribute to the development of diseases such as aging and cancer.

Epigenetics is the study of changes in gene expression patterns and activity without changes in the DNA base sequence. It has emerged as a major field of study for understanding many biological phenomena that cannot be explained by conventional genetics. DNA methylation is one of the representative mechanisms by which the structure of the epigenome may be inferred. However, the precise mechanisms of DNA methylation, gene silencing by methylation, and the target sites of DNA methylation have not yet been clearly elucidated, necessitating further research in this field.

### DISCLOSURE OF INVENTION

### Technical Problem

One aspect provides a method of detecting DNA methylation distribution in chromosomes, including: treating a prepared sample with a demethylase to remove methylated nucleotides within the nucleic acid of the sample; treating the nucleic acid, from which the methylated nucleotides have been removed, with a nuclease to modify the terminal functional groups of the nucleic acid at a site where the methylated nucleotides were removed, thereby enabling insertion of additional nucleotides; treating the nucleic acid with modified functional groups with a labeled nucleotide and a polymerase to insert the labeled nucleotide at the termini of the nucleic acid; and immunostaining the sample in which the labeled nucleotide has been inserted at the termini of the nucleic acid.

Another aspect provides a kit for detecting DNA methylation distribution, including a DNA demethylase and a labeled nucleotide, wherein the labeled nucleotide is inserted into a sample to identify methylation sites in the sample.

### Solution to Problem

One aspect provides a method of detecting DNA methylation distribution in chromosomes, including: treating a prepared sample with a demethylase to remove methylated nucleotides within the nucleic acid of the sample; treating the nucleic acid, from which the methylated nucleotides have been removed, with a nuclease to modify the terminal functional groups of the nucleic acid at the site where nucleotides were removed, thereby enabling insertion of additional nucleotides; treating the nucleic acid with the modified functional groups with a labeled nucleotide and a polymerase to insert the labeled nucleotide at the termini of the nucleic acid; and immunostaining the sample in which the labeled nucleotide has been inserted at the termini of the nucleic acid.

In an embodiment, the method may further include preparing the sample by fixing the isolated sample.

Fixatives for fixing the sample may include, but are not limited to, Carnoy's solution, paraformaldehyde (PFA), Formalin, Glutaraldehyde, Osmium tetroxide, Flemming solution, Osmic acid, Picric acid, Bouin solution, Dubosque-Brazil (D. B) solution, Mercuric chloride, acetone, B5 solution, Zenker's solution, Helly solution, and Heidenhain Susa solution. Preferably, the fixative may be Carnoy's solution or paraformaldehyde (PFA).

The fixation may be performed for a period of 1 hour to 24 hours.

Preferably, the sample may be fixed by attachment to a slide glass.

In an embodiment, the sample used may be a cell derived from a eukaryotic organism. Preferably, the sample may be an animal or plant cell. The cell may be a cell having various DNA methylation patterns, such as CG, CHG, and CHH (where H=A, T, or C), in addition to CG methylation patterns.

The animal cell may be derived from a human, cow, horse, pig, dog, sheep, goat, cat, or mouse, but is not limited thereto. The animal cell may be a kidney cell, immune cell, cancer cell, heart cell, nerve cell, beta cell (β stem cell), or fibroblast cell but is not limited thereto.

The plant cell may be a cell of cereals, legumes, tubers, vegetables, fruits, nuts, special crops, medicinal crops, monopoly crops, flowering plants, mushrooms, and the like, but is not limited thereto. The cereals include wheat, rice, corn, barley, millet, foxtail millet, sorghum, and the like; the legumes include beans, red beans, mung beans, and the like; the tubers include potatoes, sweet potatoes, and the like; the vegetables include Chinese cabbage, spinach, water dropwort, and the like; the fruits include apples, peaches, grapes, and the like; the nuts include persimmons, tangerines, walnuts, and the like; the special crops include rapeseed, sesame, peanuts, and the like; the medicinal crops include goji berries (Lycium chinense), danggui (Korean angelica), milkvetch root (Astragalus membranaceus), magnolia berries (Schisandra chinensis), and the like; the monopoly crops include tobacco, ginseng, and the like; the flowering plants include chrysanthemums, roses, lilies, and the like; and the mushrooms may include button mushrooms, wood ear mushrooms, oyster mushrooms, shiitake mushrooms, and the like.

In an embodiment, when the sample has a cell wall, the treatment with a solution containing a cell wall-degrading enzyme to degrade the cell wall may be further included. The cell wall-degrading enzyme may be cellulase, pectinase, xylanase, polygalacturonase, pectinesterase, or β-galactosidase, but is not limited thereto. Preferably, the cell wall-degrading enzyme may be cellulase.

In the present specification, “demethylase” may refer to an enzyme that catalyzes a reaction that removes a methyl group (—CH₃) from a nucleic acid including RNA, a protein, or other molecules. Preferably, the demethylase may be a DNA demethylase.

The demethylase may be selected from the group consisting of DEMETER (DME), Histone lysine demethylases (e.g., KDM1A, KDM1B, KDM2A, KDM2B, KDM3A, KDM3B, KDM3C, KDM4A, KDM4B, KDM4C, KDM4D, KDM4E, KDM4F, KDM5A, KDM5B, KDM5C, KDM5D, KDM6A, KDM6B, or KDM6C), protein-glutamate methylesterase, Jumonji, and TET/TDG, but is not limited thereto. Preferably, the demethylase may be DEMETER (DME).

DEMETER (DME) is a DNA demethylase derived from Arabidopsis thaliana, which is an enzyme capable of specifically recognizing and removing 5-methylcytosine using a DNA glycosylase domain. DEMETER may remove 5-methylcytosine present in double-stranded DNA and simultaneously create a single strand break, thereby mediating base excision repair. DEMETER may cleave the sugar-phosphate backbone to generate a phosphate group at the 3′ end.

In the present specification, the term “nucleic acid” includes polynucleotides, oligonucleotides, DNA, RNA, and analogs and derivatives thereof, for example, peptide nucleic acid (PNA) or mixtures thereof. Furthermore, the nucleic acid may be single-stranded or double-stranded, and may include molecules including polypeptides, mRNA, microRNA, or siRNA.

The methylated nucleotide may be 5-methylcytosine.

In the present specification, the term “nuclease” refers to an enzyme that catalyzes the hydrolysis of nucleic acids or nucleotides/nucleosides, which are degradation products of nucleic acids. The nuclease may be an endonuclease, exonuclease, ribonuclease, or deoxyribonuclease. Preferably, the nuclease may be an endonuclease.

The endonuclease may be an AP endonuclease, RecBCD endonuclease, T7 endonuclease, T4 endonuclease, Bal31 endonuclease, micrococcal nuclease, S1 nuclease, EcoRI, EcoRV, or Cas9 endonuclease. Preferably, the endonuclease may be an AP endonuclease.

AP endonuclease is an enzyme involved in base excision repair (BER) and may recognize apurinic/apyrimidinic sites (AP sites). The AP endonuclease may remove the 3′ terminal phosphate group generated by the above-described DEMETER and form a 3′-OH (hydroxyl) group at the 3′ end. The AP endonuclease may be Arabidopsis thaliana-derived APE1L (apurinic/apyrimidinic endonuclease 1-like), E. coli-derived Endonuclease IV, or ARP (apurinic endonuclease-redox protein), but is not limited thereto. Preferably, the AP endonuclease may be E. coli-derived Endonuclease IV.

In an embodiment, the treatment with the demethylase and the treatment with the nuclease may be performed simultaneously or sequentially. Preferably, the treatment with the demethylase and the treatment with the nuclease may be performed simultaneously. When performed simultaneously, it may be performed by preparing a mixture of the demethylase and the nuclease.

The treatment with the demethylase and nuclease mixture may be performed for a period of 10 minutes to 4 hours. Preferably, the treatment with the demethylase and nuclease mixture may be performed for 30 minutes to 3 hours. More preferably, the treatment with the demethylase and nuclease mixture may be performed for 30 minutes to 2 hours.

In an embodiment, the label may be, but is not limited to, a substance that emits fluorescence, phosphorescence, chemiluminescence, or radioactivity. The label may be applied using various methods commonly practiced in the technical field to which the present disclosure belongs. For example, the labeling may be performed by nick translation, random priming (Multiprime DNA labeling systems booklet, “Amersham” (1989)), and kinase methods (Maxam & Gilbert, Methods in Enzymology, 65:499 (1986)). The label provides a detectable signal by fluorescence, radioactivity, phosphorescence, colorimetry, gravimetry, X-ray diffraction or absorption, magnetism, enzymatic activity, mass analysis, binding affinity, hybridization radio frequency, or nano-crystals. Preferably, the label may be a fluorescent substance.

The fluorescent substance may be any one selected from the group consisting of fluorescein, fluorescein chlorotriazinyl, fluorescein isothiocyanate (FITC), rhodamine green, rhodamine red, tetramethylrhodamine, Oregon green, Alexa Fluor, JOE, ROX, HEX, Texas Red, TET, TRITC, TAMRA, cyanine dyes, and thiadicarbocyanine dyes, but is not limited thereto. Preferably, the label may be a cyanine dye. The cyanine dye may be cy3, cy3.5, cy5, cy5.5, cy7, ICG, Cypate, ITCC, NIR820, NIR2, IRDye78, IRDye80, or IRDye82. More preferably, the label may be cy3.

In an embodiment, the polymerase may be phi29 polymerase, Bst polymerase, T4 DNA polymerase, T7 DNA polymerase, Vent DNA polymerase (New England BioLabs), Pwo polymerase, Pfu polymerase, Pol I, Pol II, Pol B, Pol α, γ, δ, ε, Klenow fragment, Taq DNA polymerase, Tth polymerase, Tli polymerase, DEEPVENT polymerase, EXTaq polymerase, LA-Taq polymerase, Sso polymerase, Poc polymerase, Pab polymerase, Mth polymerase, ES4 polymerase, Tru polymerase, Tac polymerase, Tne polymerase, Tma polymerase, Tea polymerase, Tih polymerase, Tfi polymerase, Platinum Taq polymerase, Tbr polymerase, Tfl polymerase, Pfutubo polymerase, Pyrobest polymerase, KOD polymerase, or Sac polymerase, but is not limited thereto. Preferably, the polymerase used in the amplification may be the Klenow fragment (3′→5′ exo-).

The Klenow fragment (3′→5′ exo-) is an enzyme that synthesizes DNA complementary to a template strand in the 5′ to 3′ direction using dNTPs as substrates. It is a DNA polymerase in which the N-terminal domain of E. coli DNA polymerase I is removed to eliminate the 5′ to 3′ and 3′ to 5′ exonuclease activity, and it is known to be widely used in DNA sequencing.

The treatment with the labeled nucleotide and polymerase may be performed for a period of 10 minutes to 4 hours. Preferably, the treatment with the labeled nucleotide and polymerase may be performed for a period of 30 minutes to 3 hours. More preferably, the treatment with the labeled nucleotide and polymerase may be performed for a period of 30 minutes to 2 hours.

The labeled nucleotide may be cytosine to which Cy3 is attached (Cy3-dCTP).

In the treatment with the nuclease or polymerase, magnesium chloride (MgCl₂) or magnesium sulfate (MgSO₄) may be co-administered as a cofactor to increase the activity of the enzyme reaction. The cofactor may be added in an amount sufficient to achieve the desired reaction.

The treatment with the demethylase, nuclease, or polymerase may be performed at 20 °C to 40 °C to carry out the enzyme reaction. Preferably, the reaction temperature may be 36 °C to 38 °C.

The method may further include, during the treatment with the demethylase, nuclease, or polymerase, subjecting the reaction to a high temperature or treatment with a reaction stop solution to terminate the enzyme reaction. Preferably, the method may further include subjecting the reaction to 65 °C. The reaction at 65 °C may be performed for 5 minutes to 60 minutes. Preferably, the reaction at 65 °C may be performed for 5 minutes to 50 minutes. Preferably, the reaction at 65 °C may be performed for 5 minutes to 45 minutes. Preferably, the reaction at 65 °C may be performed for 10 minutes to 40 minutes. Preferably, the reaction at 65 °C may be performed for 10 minutes to 20 minutes.

After performing each step of the method, a process of washing the sample may be optionally included to remove the solution used in each step prior to proceeding to the next step. Preferably, PBS or distilled water may be used to wash the sample.

In an embodiment, “immunostaining” may refer to a staining technique based on an antigen-antibody reaction that relies on non-covalent bonds. The immunostaining may be 4′,6-diamidino-2-phenylindole (DAPI), fluorescein isothiocyanate (FITC), tetramethylrhodamine (TRITC), Alexa Fluor, phycoerythrin (PE), peridinin chlorophyll (PerCP), allophycocyanin (APC), propidium iodide (PI), avidin-biotin complex (ABC), fluorescence in situ hybridization (FISH), horseradish peroxidase (HRP), alkaline phosphatase (AP), or Cy-chrome, but is not limited thereto.

The immunostaining may be used for staining chromosomes, cell nuclei, intracellular fibrous proteins, or DNA-binding proteins, but is not limited thereto.

The methylated DNA detection method allows simultaneous detection of methylated DNA and immunostaining, thereby facilitating identification of chromatin regions as well as the density and location of methylated DNA.

According to the present invention, a sample in which DNA methylation sites are labeled and immunostained may be stably stored for a long period and may be repeatedly observed in the same manner.

Such observation may be carried out using a microscope.

In the present specification, the term “DNA methylation” refers to the attachment of a methyl group to a base that makes up DNA. DNA methylation generally refers to a reaction in which a methyl group is transferred to the 5-carbon position of cytosine in DNA by a DNA methyltransferase, thereby converting it into 5-methylcytosine (5mC). Because DNA methylation primarily induces the formation of heterochromatin, which is accompanied by chromatin condensation, it is known that chromosome regions with high levels of DNA methylation exhibit a heterochromatin structure, whereas regions with lower levels of DNA methylation exhibit a euchromatin structure. DNA methylation plays a critical role in the development, differentiation, and progression of eukaryotic organisms, and when normal DNA methylation does not occur, it may lead to aging and diseases such as cancer.

In the present specification, the term “detection” may refer to measuring and confirming whether the target substance (i.e., methylated DNA in this disclosure) is present in a sample, or determining the location of the target substance within the sample. In addition, in the present specification, “search” and “detection” may sometimes be used interchangeably.

This DNA methylation distribution detection method may be capable of qualitatively detecting the density of methylated nucleotides in a sample, based on the extent to which labeled nucleotides are inserted and express the label.

Another aspect provides a kit for detecting DNA methylation distribution, including a DNA demethylase and a labeled nucleotide, wherein the labeled nucleotide is inserted into a sample to identify methylation sites in the sample.

In an embodiment, the kit may further include one or more of a reagent and carrier, a washing buffer, a sample diluent, an enzyme, a substrate, a reaction stop solution, and instructions for use.

The kit may further include an immunostaining reagent for staining the nucleus, microtubules, fibrous proteins, or DNA-binding proteins of the sample.

The kit may further include a fixative for fixing the sample and a substrate to which the sample may be attached.

### Advantageous Effects of Invention

According to one aspect, the method of detecting DNA methylation using enzymes can accurately identify the location and density of 5-methylcytosine present in chromosomes, and can also be advantageously applied to identifying chromatin regions and performing epigenetic analyses by employing various conventional immunofluorescence labeling techniques.

### BRIEF DESCRIPTION OF DRAWINGS

FIG. 1 schematically illustrates an experimental scheme.

FIG. 2 is a photograph confirming the DNA methylation detection method using prophase I chromosomes of Brassica rapa.

FIG. 3 shows observation of meiotic chromosomes of Brassica rapa specifically labeled with a fluorescent substance using a DNA demethylase.

FIG. 4 shows identification of DNA methylation regions in interphase of Brassica rapa meiotic cells.

FIG. 5 shows meiotic chromosomes of Capsicum annuum specifically labeled with a fluorescent substance using a DNA demethylase.

FIG. 6 shows observation of heterochromatin in meiotic chromosomes by simultaneously treating with a DNA methylation labeling method using a DNA demethylase and an immunofluorescence method of detecting histone modifications.

FIG. 7 shows identification of distribution patterns of DNA methylation and microtubules in mitotic metaphase chromosomes of Allium cepa.

### BEST MODE FOR CARRYING OUT THE INVENTION

Hereinafter, preferred Examples are presented to aid in understanding the present disclosure. However, the following Examples are provided only to facilitate understanding of the present disclosure, and the content of the present disclosure is not limited by the following Examples. The Examples may be variously modified, and the Examples are not limited to the Examples disclosed below, but may be implemented in various forms.

### Example 1. DNA Chromosome Painting Technique

### 1.1. Methylated DNA Staining Method

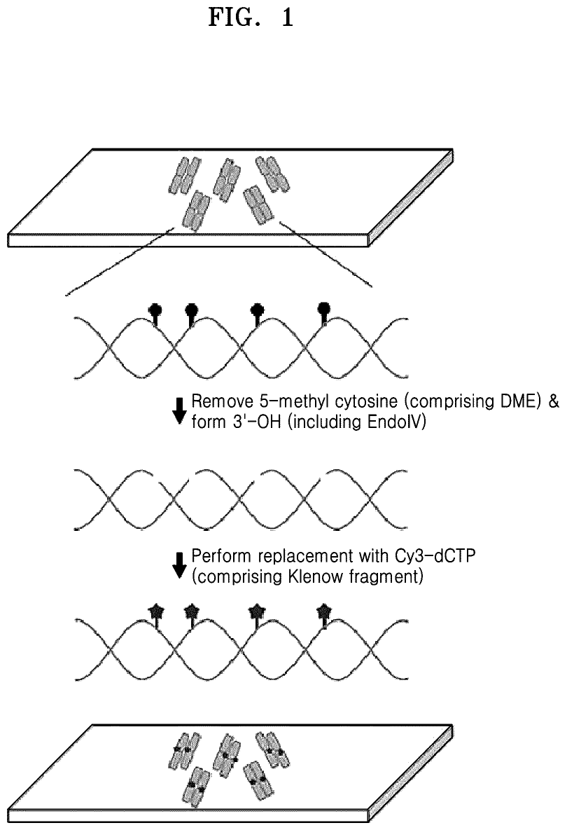

To stain methylated DNA, samples were fixed using Carnoy's solution. The chromosomes of the fixed sample were spread on a slide glass and fixed, after which DNA demethylase (1 μg to 3 μg) and AP endonuclease (10 U) were directly applied onto the slide in a DNA demethylase reaction buffer (50 mM NaCl, 10 mM Tris-HCl pH 7.4, 0.5 mM DTT). The reaction was performed at 37 °C for 1 hour to remove 5-methylcytosine and induce the formation of a hydroxyl group (—OH) at the 3′ end. Subsequently, the sample was treated at 65 °C for 15 minutes to terminate the DNA demethylase/AP endonuclease enzyme reaction. After the enzyme reaction was stopped, the slide was washed with PBS buffer, and then fluorescently labeled nucleotide (Cy3-dCTP, 500 pmole) and Klenow fragment (3′→5′ exo-) (10 U) as DNA polymerase were applied onto the slide under a DNA demethylase reaction buffer containing 2.5 mM MgCl₂. The sample, in which the removed 5-methylcytosine position was replaced with Cy3-labeled cytosine, was treated at 65 °C for 15 minutes to terminate the DNA polymerase reaction. Subsequently, the slide was washed with PBS buffer, and the chromosomes were stained with DAPI solution or other immunofluorescence staining. The labeled chromosomes were observed under a fluorescence microscope, and the distribution of 5-methylcytosine was identified through comparison with the entire DAPI-stained chromosomes. The methylated DNA staining method is schematically illustrated in FIG. 1.

### 1.2. Confirmation of Methylated DNA Staining

To confirm whether 5-methylcytosine was replaced with fluorescently labeled cytosine, the method of Example 1.1 was performed using a Brassica rapa sample.

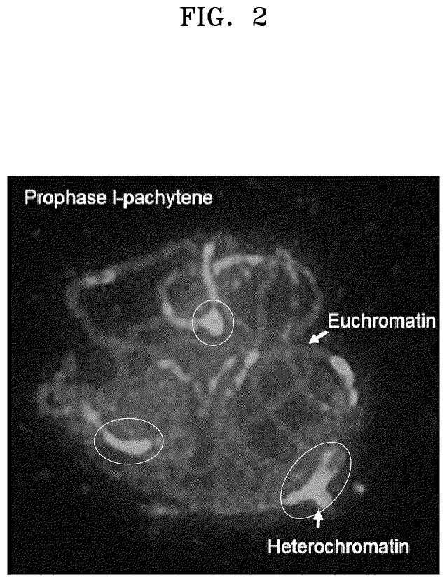

FIG. 2 is a photograph confirming the DNA methylation detection method using prophase I chromosomes of Brassica rapa.

As shown in FIG. 2, in the DAPI-stained Brassica rapa sample, regions with high Cy3 signals (indicated by white circles), which are specific fluorescent labels for 5-methylcytosine, were observed. The regions where the Cy3 signal was hardly detected and the DAPI signal was predominantly visible were euchromatin with a low degree of DNA methylation, and the regions with high Cy3 signals were confirmed to be heterochromatin. Therefore, it was confirmed that this method may be used to identify DNA methylation sites and chromatin regions.

Experimental Example 1. DNA Methylation Pattern Labeling Using DNA Demethylase in Meiotic Chromosomes with Euchromatin Structure

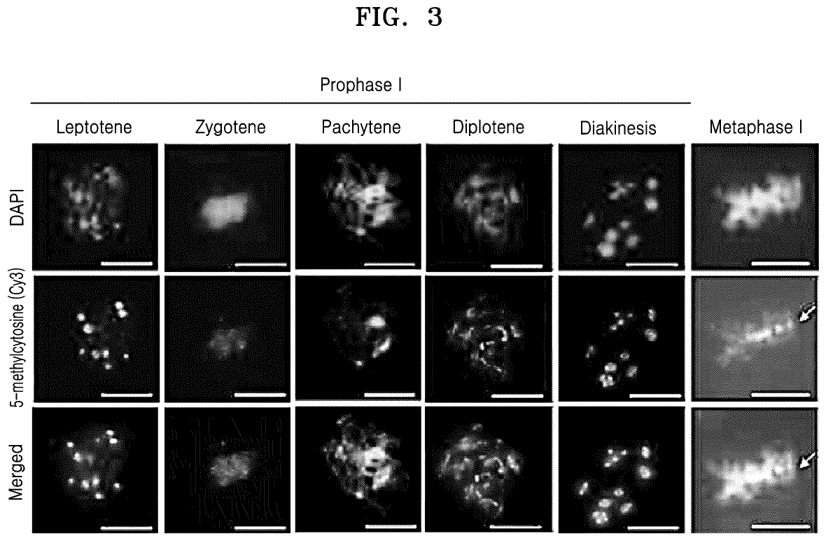

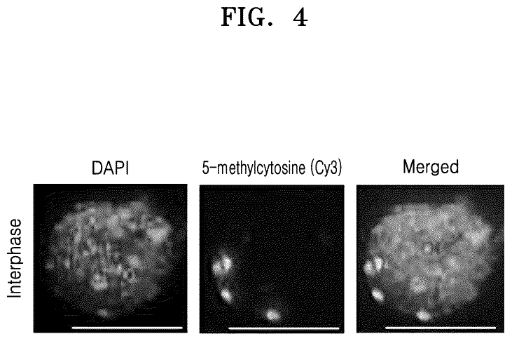

For DNA methylation profiling in euchromatin, meiotic chromosomes of Brassica rapa, which has a high proportion of euchromatin in the plant genome, were stained by the method of Example 1.1 to confirm the DNA methylation distribution. The results are shown in FIGS. 3 and 4.

FIG. 3 shows observation of meiotic chromosomes of Brassica rapa specifically labeled with a fluorescent substance using a DNA demethylase.

FIG. 4 shows identification of DNA methylation regions in interphase of Brassica rapa meiocytes.

As shown in FIG. 3, it was confirmed that Cy3 was emitted at specific regions of the prophase I and metaphase I chromosomes, confirming that DNA methylation detection is possible. In particular, the positions where the Cy3 signal was specifically high in metaphase (white arrows) were identified as centromeres composed of heterochromatin, confirming that Cy3-cytosine was specifically labeled at heterochromatin regions where hypermethylation occurs.

As shown in FIG. 4, in the interphase of Brassica rapa chromosomes, the Cy3 signal was observed at the nuclear periphery. This is the same result as known that heterochromatin is clustered at the nuclear periphery, and through this, it was confirmed that the location and structure of heterochromatin in the nucleus may be identified.

### Experimental Example 2. DNA Methylation Pattern Labeling Using DNA Demethylase in Meiotic Chromosomes With Heterochromatin Structure

To confirm the DNA distribution pattern in heterochromatin, DNA methylation staining was performed using meiotic chromosomes of Capsicum annuum, which has a high proportion of heterochromatin in the plant genome.

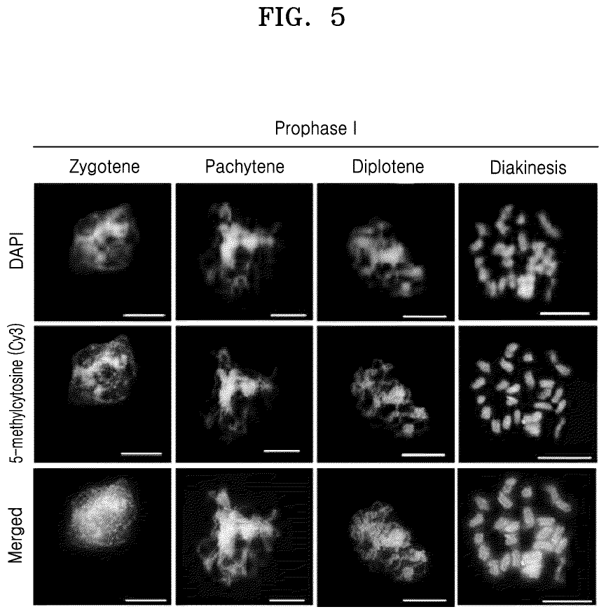

FIG. 5 shows meiotic chromosomes of Capsicum annuum specifically labeled with a fluorescent substance using a DNA demethylase.

As shown in FIG. 5, in the case of Capsicum annuum, fluorescence labeling with Cy3-cytosine was confirmed throughout the chromosomes. The Capsicum annuum genome is known to have a high proportion of repetitive sequences and transposable elements that induce heterochromatin structure, and it was confirmed that the location and structure of such heterochromatin may be identified through DNA methylation staining.

### Experimental Example 3. Simultaneous Observation of Histone Protein Modification and DNA Methylation Patterns by Combining Immunofluorescence Labeling and DNA Methylation Pattern Labeling Using DNA Demethylase

To confirm histone protein modification, which is one of the representative epigenetic factors that affect chromosome structure along with DNA methylation, the following method was performed. Specifically, meiotic chromosomes of Brassica rapa were specifically labeled with a fluorescent substance for DNA methylation by the method of Example 1.1, and then immunofluorescence labeling was performed using an antibody that detects H3K9me2, a histone modification representative of heterochromatin, to observe the epigenome profile in the chromosomes.

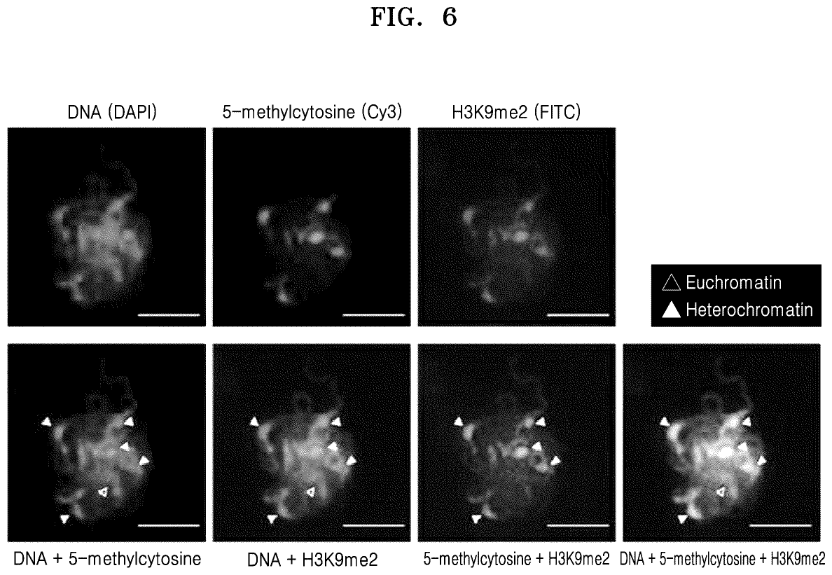

FIG. 6 shows the observation of DNA methylation and histone modification patterns by simultaneously applying a DNA methylation pattern labeling method using a DNA demethylase and an immunofluorescence technique in Brassica rapa chromosomes.

As shown in FIG. 6, it was observed that the hypermethylated regions and the regions where H3K9me2 was present mostly overlapped in the chromosomes of meiosis I prophase. The regions where the two signals overlapped were expected to be regions with constitutive heterochromatin. On the other hand, regions where DNA staining (DAPI) was darker compared to the signals corresponding to DNA methylation and H3K9me2 were expected to be euchromatin.

From the above results, it was confirmed that multiple immunofluorescence staining is possible after methylated DNA staining, and that DNA methylation and histone modification may be observed simultaneously, suggesting the possibility of a new concept of epigenome profiling technique.

### Experimental Example 4. Multiplexed Fluorescence Labeling Combined With Immunofluorescence Labeling Method

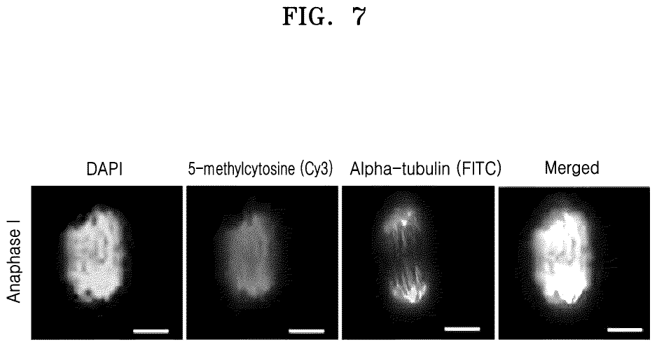

To confirm whether it is possible to detect microtubules, which dynamically change along with chromosomes during cell division, by immunofluorescence labeling, DNA methylation and microtubule patterns were observed together. Specifically, Allium cepa root tip meristem tissue was fixed with paraformaldehyde (PFA), DNA methylation was confirmed by the method of Example 1.1, and then two types of immunofluorescence staining, DAPI and FITC, were performed. The results are shown in FIG. 7.

FIG. 7 shows the confirmation of the distribution patterns of DNA methylation and microtubules in mitotic metaphase chromosomes of Allium cepa.

As shown in FIG. 7, it was confirmed that DNA was simultaneously labeled with DAPI, DNA methylation sites with Cy3, and microtubules (a-tubulin) with FITC. In addition, it was confirmed that the DNA methylation pattern labeling method using a DNA demethylase was effective in chromosomes fixed using not only Carnoy's solution but also paraformaldehyde (PFA).

From the above results, it was confirmed that multiplexed fluorescence labeling is possible through various fluorescent stains after DNA methylation staining, enabling DNA methylation distribution analysis and simultaneous analysis of related proteins, and that DNA methylation labeling is possible even when using various fixatives.

The foregoing description of the present disclosure is for illustrative purposes, and those of ordinary skill in the art to which the present disclosure pertains will understand that the present disclosure may be readily modified in other specific forms without changing the technical spirit or essential features of the present disclosure. Therefore, the Examples described above should be understood to be illustrative in all respects and not restrictive.

1. A method of detecting DNA methylation distribution in a chromosome, the method comprising:
treating a prepared sample with a demethylase to remove a methylated nucleotide within a nucleic acid of the sample;
treating the nucleic acid, from which the methylated nucleotide has been removed, with a nuclease to modify a terminal functional group of the nucleic acid at a site where the methylated nucleotide has been removed, wherein the modification allows for insertion of an additional nucleotide;
treating the nucleic acid with the modified terminal functional group with a labeled nucleotide and a polymerase to insert the labeled nucleotide at the terminal functional group of the nucleic acid; and
immunostaining the sample in which the labeled nucleotide has been inserted at the terminal functional group of the nucleic acid.

2. The method of claim 1, further comprising preparing the sample by fixing an isolated sample.

3. The method of claim 1, wherein the sample is a cell derived from a eukaryotic organism.

4. The method of claim 1, wherein the demethylase is a DNA demethylase.

5. The method of claim 1, wherein the methylated nucleotide is 5-methylcytosine.

6. The method of claim 1, wherein the nuclease is an endonuclease.

7. The method of claim 1, wherein the modification of the terminal functional group of the nucleic acid comprises removing a 3′ terminal phosphate group at the site where the methylated nucleotide has been removed, and
forming a hydroxyl group at the 3′ end where the phosphate group has been removed.

8. The method of claim 1, wherein the treatment with the demethylase and the treatment with the nuclease are performed simultaneously or sequentially.

9. The method of claim 1, wherein the label is attached using a substance that emits fluorescence, phosphorescence, chemiluminescence, or radioactivity.

10. The method of claim 1, wherein the method is capable of qualitatively detecting a density of the methylated nucleotides in the immunostained sample based on an extent to which the labeled nucleotides are inserted and express the label.

11. The method of claim 1, wherein the immunostaining is performed by one or more selected from the group consisting of 4′,6-diamidino-2-phenylindole (DAPI), fluorescein isothiocyanate (FITC), tetramethylrhodamine (TRITC), Alexa Fluor, phycoerythrin (PE), peridinin chlorophyll (PerCP), allophycocyanin (APC), propidium iodide (PI), avidin-biotin complex (ABC), fluorescence in situ hybridization (FISH), horseradish peroxidase (HRP), alkaline phosphatase (AP), and Cy-chrome.

12. The method of claim 1, wherein the immunostaining stains one or more selected from chromosomes, cell nuclei, intracellular fibrous proteins, and DNA-binding proteins of the sample.

13. A kit for detecting DNA methylation distribution, comprising:
a demethylase capable of removing methylated nucleotides from a nucleic acid of a sample;
a nuclease capable of modifying a functional group of the nucleic acid of the sample to allow for insertion of an additional nucleotide;
a labeled nucleotide capable of being inserted into the nucleic acid at a position from which a methylated nucleotide has been removed;
a polymerase capable of causing the labeled nucleotide to be inserted into the nucleic acid in the sample; and
an immunostaining reagent capable of staining a chromosome, a cell nucleus, an intracellular fibrous protein, or a DNA-binding protein in the sample.

14. The kit of claim 13, further comprising a fixative for fixing the sample and a substrate to which the sample can be attached.

15. The kit of claim 13, wherein the kit is capable of detecting methylation distribution in the sample by identifying the location and degree of expression of the label in the sample stained with the immunostaining reagent.
